(19) United States
(12) Patent Application Publication
(10) Pub. No.: US 2020/0179479 A1
(43) Pub. Date: Jun. 11, 2020

(54) INHIBITION OF ALPHA5BETA1 INTEGRIN WITH ATN-161 AS A NOVEL THERAPY FOR VASCULAR DEMENTIA

(71) Applicant: University of Kentucky Research Foundation, Lexington, KY (US)

(72) Inventors: Gregory Jaye Bix, New Orleans, LA (US); Jill Marie Roberts, Lexington, KY (US)

(21) Appl. No.: 16/575,248

(22) Filed: Sep. 18, 2019

Related U.S. Application Data
(60) Provisional application No. 62/732,917, filed on Sep. 18, 2018.

(51) Int. Cl. A61K 38/08; A61P 25/28

(52) U.S. Cl. CPC A61K 38/08; A61P 25/28

(57) ABSTRACT

Disclosed herein are methods for treating or preventing a hypoperfusion related disease in a subject in need thereof, comprising identifying a subject with cerebral hypoperfusion and administering an effective amount of an α5β1 integrin inhibitor to the subject. Methods for decreasing α5β1 expression in the cerebrovasculature, comprising: administering an effective amount of an α5β1 integrin inhibitor to a subject in need thereof are also disclosed. A method for preventing or decreasing the risk of developing diabetes related dementia, comprising: identifying a subject with diabetes and administering an effective amount of an α5β1 integrin inhibitor to the subject is further disclosed.

### RELATED APPLICATIONS

This application claims priority from U.S. Provisional Patent Application No. 62/732,917 filed on Sep. 18, 2018, the entire disclosure of which is incorporated herein by this reference.

### GOVERNMENT INTEREST

This invention was made with government support under grant number R01NS065842 awarded by the National Institutes of Health. The government has certain rights in the invention.

### TECHNICAL FIELD

The present invention relates to inhibitors of α5β1 integrin as therapeutics for the prevention or treatment of vascular dementia.

### BACKGROUND AND SUMMARY

Previous studies have shown that the domain V (DV) protein fragment of the brain extracellular matrix (ECM) proteoglycan perlecan is neuroprotective and neuroreparative (pro-angiogenic) following experimental ischemic stroke. This may occur in part through the generation of vascular endothelial growth factor (VEGF). Prior studies further demonstrated that this occurs by DV binding to a particular fibronectin brain endothelial cell receptor, α5β1 integrin. Notably, brain endothelial cell α5β1 integrin expression is tightly regulated, primarily expressed during development, following an injury such as ischemic stroke. To investigate the link between stroke-induced upregulation of α5β1 integrin and the therapeutic effects of DV, transient middle cerebral artery occlusion (MCAo) in mice that have endothelial cell-selective knockout of the α5 integrin (referred to hereafter as α5KO) was performed. There was little to no discernible infarct or neuronal injury following MCAo. Importantly, no differences in cerebrovascular anatomy or collateral blood flow were noted in these mice that could account for this difference in ischemic injury. One of the prominent ways that ischemic injury expands is via disruption of the blood-brain barrier (BBB), allowing for increased edema and infiltration of inflammatory cells. In fact, the BBB appears to open and close repeatedly after injury. One aspect of the present invention is that α5β1 integrin is increased over time following MCAo and that blockade of α5β1 integrin (via a small peptide ATN-161) leads to decreased infarct volumes (FIG. 1A-FIG. 1B), decreased edema, decreased leukocyte infiltration, and improved functional outcome. In addition, α5KO mice demonstrate improved BBB integrity through increased expression of the TJ protein claudin-5 and decreased leakage of IgG into brain parenchyma following stroke compared to control mice.

Vascular pathology is now recognized as a prominent cause of dementia behind Alzheimer's disease. Vascular contributions to cognitive impairment and dementia (VCID) may occur, at least in part, as a result of chronic cerebral hypoperfusion, and eventually leads to white matter lesions, BBB disruptions and cognitive impairment. The altered cerebral perfusion that underlies VCID may result in a compensatory increase in new blood vessels generated from pre-existing blood vessels, a process known as angiogenesis. The BBB formed by pre-existing vasculature breaks down during the earliest stages of angiogenesis, when activated endothelial cells dislodge their cell-cell contacts (i.e. tight junction proteins including claudin-5), and proteolytically degrade surrounding basement membranes; this may contribute to brain dysfunction. Bilateral carotid artery stenosis (BCAS) is one experimental model of chronic hypoperfusion, using micro-coils wrapped around the carotid arteries to decrease, but not eliminate, blood flow to the brain. Studies have shown that BCAS leads to decreased cerebral blood flow (FIG. 2A-FIG. 2B), and within 30 days post-stenosis increased BBB disruptions, inflammation and cognitive impairment is observed in mice⁹⁻¹³. It was recently published that many of these changes occur at much earlier time points following BCAS in both the white and grey matter.

Intriguingly, α5β1 integrin endothelial signaling has also been found to contribute to the myogenic response in which arterioles and small arteries either constrict or dilate in response to increased or decreased intraluminal pressure, respectively. This suggests the possibility that α5β1 integrin may also contribute to compensatory, but ultimately aberrant cerebrovascular vasodilation in the BCAS model, a process that also contributes to abnormal cerebrovascular blood flow, remodeling, and BBB integrity.

In addition, as common co-morbid conditions are significant risk factors for developing dementia, it is important to examine how such co-morbidities could impact α5β1 integrin expression and/or the therapeutic potential of α5β1 integrin blockade. One particular risk factor for dementia is diabetes mellitus, as diabetes promotes degeneration of the vascular system and diminishes its regenerative capacity. Indeed, diabetic patients are twice as likely to develop AD or other types of dementias in comparison with those that had normal blood sugar control. Rodent studies have found that diabetes impairs collateral flow compensation, increases neuronal cell death, increases inflammation, exacerbates white matter lesions, increases BBB permeability, and worsens cognitive impairment compared to control animals in various models of hypoperfusion including BCAS. However, very little is known about how diabetes leads to increased BBB permeability during chronic cerebral hypoperfusion, and whether α5β1 integrin could play a role in diabetic cerebrovascular pathology. Genetically altered leptin resistant db/db mouse (referred to hereafter as DB), which produces a phenotype of hyperglycemia and obesity within 3 months of age with vascular pathologies having been reported after 5 months of age²⁵ were used in experiments.

Acute treatment for ischemic stroke is currently limited to recanalization/reperfusion strategies only provided to a minority of patients: intravenous tissue plasminogen activator (t-PA) and/or endovascular thrombectomy. While these treatments remove the blood vessel-obstructing thrombus, they fail to impact secondary reperfusion injury. Reperfusion injury results from an influx of factors, including calcium, cytokines, reactive oxygen species, growth factors, and matrix metalloproteinases (MMPs), that induce extracellular matrix (ECM) degradation, leukocyte infiltration, and apoptotic cascades. These processes destabilize the blood-brain barrier (BBB), a vascular defense mechanism composed of non-fenestrated endothelial cells, tightly bound by tight junction (TJ) proteins and junctional adhesion molecules (JAMs) that are surrounded by astrocytic endfeet. Collectively, the BBB breakdown contributes to the expansion of the infarct volume from the site of initial injury (core) into the surrounding at risk tissue (peri-infarct). Therefore, targeting reperfusion injury could alter ischemic stroke outcomes.

One potential target is the integrin receptor family, a group of cell surface transmembrane glycoprotein receptors for the ECM. They are composed of non-covalently bound a and 3 subunits. Vital to stroke pathophysiology, integrins have been implicated in cell migration, cellular adhesion, and cellular survivability. One particular integrin, integrin α5β1, is of vital interest due to its increased expression in hypoxia and stroke models, while exhibiting variable roles in tumor growth and metastisis¹¹, atherosclerosis development, and promotion of wound healing. Previous data has shown that elimination of the specific pro-angiogenic integrin α5β1 in endothelial specific knockout (α5KO) mice results in significant resistance to ischemic stroke injury. Furthermore, these α5KO mice maintained near-baseline pre-stroke levels of the TJ protein claudin-5 and exhibited little to no disruption of the BBB barrier. These results, combined with previous studies demonstrating a sustained increase in the post-stroke expression of integrin α5β1, suggest that inhibition of α5β1 integrin could be therapeutic by stabilizing the BBB. Early intervention of the BBB following ischemic stroke is ideal, as its role in injury is not immediately apparent. To that end, in the present invention, in one embodiment, the small peptide non-competitive integrin α5β1 inhibitor, ATN-161, in a mouse model of ischemic stroke. Importantly, ATN-161 was safe and well-tolerated in a number of Phase I and II clinical oncology studies, supporting its potential as a ‘shovel-ready’ clinical stroke therapeutic.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1A shows ATN-161 (1 mg/kg) reduces infarct volume following MCAo. Representative images of T2 weighted MRI scans on PSD 3 show white wedge-shaped cortical infarct in vehicle group and a reduction in the ATN-161 group. Vehicle: n=7; ATN-161: n=8; *p≤0.05 between groups.

FIG. 1B shows ATN-161 (1 mg/kg) reduces infarct volume following MCAo. Infarct volume analysis of T2 weighted MRI scan. Vehicle: n=7; ATN-161: n=8; *p≤0.05 between groups.

FIG. 2A shows Cerebral blood flow is decreased after BCAS. Representative laser Speckle images of skull surface vessels prior to (left) and 15 min after (right) BCAS indicate decreased blood flow.

FIG. 2B. shows Cerebral blood flow is decreased after BCAS. Quantification of region of interest. Baseline: n=6; BCAS: n=6; ***p≤0.001.

FIG. 3A shows Treatment with ATN-161 decreases α5 expression compared to controls following BCAS (14 days). Representative images within the cortex of α5 staining in sham animals, Sham: n=4

FIG. 3B shows Treatment with ATN-161 decreases α5 expression compared to controls following BCAS (14 days). Representative images within the cortex of α5 staining in animals treated with vehicle. BCAS: n=8

FIG. 3C shows Treatment with ATN-161 decreases α5 expression compared to controls following BCAS (14 days). Representative images within the cortex of α5 staining in BCAS animals treated with ATN-161 (1 mg/kg; i.p.). ATN-161 n=4

FIG. 3D shows Treatment with ATN-161 decreases α5 expression compared to controls following BCAS (14 days). Quantification of α5-positive pixel density. ***p≤0.001 compared to sham group.

FIG. 4A shows treatment with ATN-161 decreases inflammation compared to controls following BCAS (14 days). Representative images within the striatum of GFAP (astrocyte marker) staining in sham animals, Sham: n=4

FIG. 4B shows treatment with ATN-161 decreases inflammation compared to controls following BCAS (14 days). Representative images within the striatum of GFAP (astrocyte marker) staining in BCAS animals treated with vehicle, BCAS: n=8

FIG. 4C shows treatment with ATN-161 decreases inflammation compared to controls following BCAS (14 days). Representative images within the striatum of GFAP (astrocyte marker) staining in BCAS animals treated with ATN-161 (1 mg/kg; i.p.); treatment immediately after surgery and on post-stenosis days 2, 4, 6, 8, 10, 12. ATN-161: n=4

FIG. 4D shows treatment with ATN-161 decreases inflammation compared to controls following BCAS (14 days). Quantification of GFAP-positive pixel density. **p≤0.01 compared to sham group.

FIG. 5 shows improved cognitive outcome in BCAS animals following ATN-161 treatment. On post-stenosis day 14, mice underwent y-maze testing and % spontaneous alternation (working memory) was quantified. BCAS animals were treated with vehicle or ATN-161 (1 mg/kg; i.p.) treatment immediately after surgery and on post-stenosis days 2, 4, 6, 8, 10, 12. A positive trend is observed in mice treated with ATN-161. Sham: n=4; BCAS: n=8; ATN-161: n=4

FIG. 6A shows α5 expression is increased in diabetic animals compared to controls. Representative images within the cortex of α5 staining in 8 month old wildtype (WT)

FIG. 6B shows α5 expression is increased in diabetic animals compared to controls. Representative images within the cortex of α5 staining in 8 month old diabetic (DB) animals.

FIG. 6C shows Quantification of α5-positive pixel density shows a trend of an increase in α5 expression levels in both the cortex and the striatum. WT: n=4; DB: n=4.

FIG. 7 A shows ATN-161 is safe and effective following ischemic stroke. Following reperfusion, percent change of heart rate (bpm).

FIG. 7B shows ATN-161 is safe and effective following ischemic stroke. Following reperfusion, percent change of pulse distension (μM).

FIG. 7C shows ATN-161 is safe and effective following ischemic stroke. Following reperfusion, percent change of body temperature (° C.).

FIG. 7D shows ATN-161 is safe and effective following ischemic stroke. Following reperfusion, percent change of weight (g).

FIG. 7E shows Representative images of infarct volume at PSD3.

FIG. 7F shows TTC analysis of infarct volume at PSD3 (p=0.0004; Vehicle 35.4114.04, n=12; ATN-161 16.3714.25, n=12).

FIG. 7G Injection schematic.

FIG. 8 A show ATN-161 reduces MRI infarct volume and edema. Representative ADC images. Arrows indicate infarcted region.

FIG. 8B shows ADC analysis for infarct volume (p=0.0220; Vehicle 17.9±4.677, n=7; ATN-161 12.24±3.758, n=8).

FIG. 8C shows Representative T2-weighted MRI images. Arrows indicate edematous region.

FIG. 8D shows T2-weighted MRI analysis for edema volume (p=0.0097; Vehicle 15.12±3.206, n=7; ATN-161 8.777±4.658, n=8). Linear correlation of infarct volume and edema volume.

FIG. 8E shows Linear correlation of infarct volume and edema volume for all treatments (p=0.0001 R\hat{ }=0.7154).

FIG. 8F shows Linear correlation of infarct volume and edema volume for Vehicle (p=0.0786, R\hat{ }=0.4931)

FIG. 8G shows Linear correlation of infarct volume and edema volume for ATN-161 (p=0.0072, R\hat{ }2=0.0072) treated animals.

FIG. 9 A shows TN-161 reduces functional deficit and integrin α5β1 expression. Analysis of 11-point Neuroscore (p=0.0002; Vehicle 2.286±0.8284, n=14; ATN-161 3.286±0.7263, n=14; 95% Cl −0.07218 to 0.4636.)

FIG. 9B shows ATN-161 reduces functional deficit and integrin α5β1 expression. Integrin α5β1 analysis at PSD3 in peri-infarct (p=0.0835, Vehicle 308484±53485, n=7; ATN161 162166±23348, n=7).

FIG. 9C shows Integrin α5β1 analysis at PSD3 in core (p=0.3138, Vehicle 332114±209410, n=7; ATN-161 233111134950, n=7).

FIG. 9D shows Representative images for B and C. 10× magnification, scale bar=100 μm.

FIG. 10 A shows ATN-161 reduces BBB permeability following ischemic stroke. Representative images of B. 20× magnification, scale bar=50 μm.

FIG. 10B shows IgG positive pixel analysis in the core (p=0.0234; Vehicle 70125±53485, n=7; ATN-161 16753±23348, n=8).

FIG. 10C shows Linear correlation between IgG positive pixels and edema volume after Vehicle (p=0.3705, R\hat{ }=0.1621).

FIG. 10D shows Linear correlation between IgG positive pixels and edema volume after ATN-161 (p=0.0590, R\hat{ }2=0.474) administration.

FIG. 11 A shows ATN-161 stabilizes cerebrovasculature following ischemic stroke. qPCR analysis of ipsilateral cortex at PSD3 for collagen IV (p=0.0115; Sham 1.018±0.1573, n=4; Vehicle 0.3543±0.1013, n=7; ATN161 0.8563±0.6898, n=8; 95% Cl −0.6093 to −0.1379).

FIG. 11B shows Representative images of FIG. 11C 10× Magnification, scale bar=100 μm.

FIG. 11C shows Analysis for Claudin-5 positive pixels in the core at PSD3 (p=0.0270; Vehicle 19830±5538, n=5; 44220±19878, n=6).

FIG. 11D shows Representative images of FIG. 11E. 10× magnification, scale bar=100 μm.

FIG. 11E shows Analysis for fibronectin positive pixels at PSD3 in the core (p=0.5819; Vehicle 106153±183136, n=6; ATN-161 158938±164906, n=8).

FIG. 11F shows Representative images of FIG. 11G. 10× magnification, scale bar=100 μm.

FIG. 11G shows Analysis for fibronectin positive pixels at PSD3 in the peri-infarct (p=0.4665; Vehicle 28782±40979, n=6; ATN-161 48028±16730, n=8).

FIG. 12 A shows ATN-161 results is reduced MMP-9 expression that is regionally dependent. qPCR analysis of ipsilateral cortex at PSD3 for MMP-9 (p=0.0028; Sham 1.018±0.1759, n=4; Vehicle 6.761±5.321, n=7; ATN-161 2.757±2.401, n=7; 95% Cl 0.7299 to 4.232).

FIG. 12B shows Representative images of FIG. 12C. 10× magnification, scale bar=100 μm.

FIG. 12C shows Analysis for MMP-9 (green) positive pixels at PSD3 in the core (p=0.0734; Vehicle 443691±220951, n=6; ATN-161 232262±68098, n=7).

FIG. 12D shows Representative images of FIG. 12E. 10× magnification, scale bar=100 μm.

FIG. 12E shows Analysis for MMP-9 (green) positive pixels at PSD3 in the peri-infarct (p=0.0450; Vehicle 189516±170441, n=7; ATN-161 42227±180171, n=7).

FIG. 13 A shows ATN-161 reduces inflammatory cells after experimental ischemic stroke. qPCR analysis of ipsilateral cortex at PSD3 for A. IL-13 (p=0.0006; Vehicle 27.04±19.96, n=8; Sham 1.023±0.1666, n=4; p=0.0192, ATN-161 11.89±9.235, n=7 95% Cl 4.209 to 18.30).

FIG. 13B shows ATN-161 reduces inflammatory cells after experimental ischemic stroke. qPCR analysis of ipsilateral cortex at PSD3 for CXCL12 (p=0.0022; Vehicle 0.5271±0.1779, n-7; Sham 1.015±0.1964, n=4; ATN-161 0.8488±0.2846, n=8).

FIG. 13C shows Representative images of FIG. 13D. 4× Magnification, scale bar=250 μm. 20× images of representative core areas are outlined in red boxes.

FIG. 13D shows Analysis for CD45 positive pixels at PSD3 (p=0.0225; Vehicle 499674±290485, n=7; ATN-161 215451±106062, n=8).

FIG. 13E shows A visual overview of integrin α5β1's effects on the cerebrovascular unit following ischemic stroke.

FIG. 13F shows A visual overview of ATN-161's effects on integrin α5β1 and the post-stroke cerebrovasculature.

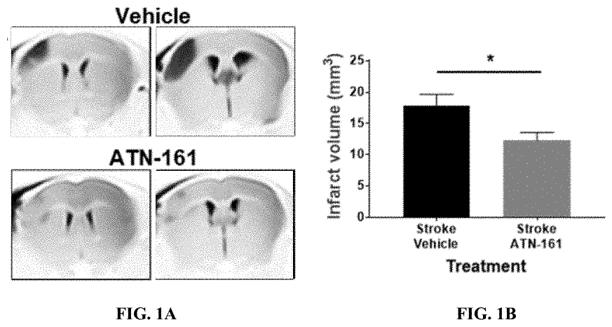

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

### Definitions

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims. Thus, for example, reference to “a device” includes a plurality of such devices, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to a target in need of a diagnosis. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, whether male or female, are intended to be covered. A subject may also be afflicted with a disease or disorder. The term “patient” may be used to specifically refer to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein. Such a diagnosis can be in reference to a disorder, such as diabetes, and the like, as discussed herein.

### EMBODIMENTS

In one embodiment, the present invention relates to a method of treating or preventing a hypoperfusion related disease in a subject in need thereof, comprising: identifying a subject with cerebral hypoperfusion and administering an effective amount of an α5β1 integrin inhibitor to the subject. In a further embodiment of the present invention, the hypoperfusion related disease is neuro inflammation, dementia, or cognitive impairment. In other embodiments of the present invention, the α5β1 integrin inhibitor is selected from: AST-161, Ac-PhScN-NH2, CRRETAWAC, or combinations thereof. In other embodiments, the α5β1 integrin inhibitor is AST-161. In some embodiments of the present invention, the cerebral hypoperfusion is chronic or acute. In a further embodiment of the present invention, the cerebral hypoperfusion fully or partially occludes vasculature. In some embodiments of the present invention, the cerebral hypoperfusion is caused by a stroke. In further embodiments of the present invention, the amount of AST-161 administered is about 1 mg/kg to about 5 mg/kg.

In further embodiments of the present invention, the administration of the α5β1 integrin inhibitor is acute and/or subacute.

Another embodiment of the present invention is a method of decreasing α5β1 expression in the cerebrovasculature, comprising: administering an effective amount of an α5β1 integrin inhibitor to a subject in need thereof. In other embodiments of the present invention, the α5β1 integrin inhibitor is selected from: AST-161, Ac-PhScN-NH2, CRRETAWAC, or combinations thereof. In other embodiments, the α5β1 integrin inhibitor is AST-161. In further embodiments of the present invention, the amount of AST-161 administered is about 1 mg/kg to about 5 mg/kg.

Another embodiment of the present invention is a method of preventing or decreasing the risk of developing diabetes related dementia, comprising: identifying a subject with diabetes and administering an effective amount of an α5β1 integrin inhibitor to the subject. In other embodiments of the present invention, the α5β1 integrin inhibitor is selected from: AST-161, Ac-PhScN-NH2, CRRETAWAC, or combinations thereof. In other embodiments, the α5β1 integrin inhibitor is AST-161. In further embodiments of the present invention, the amount of AST-161 administered is about 1 mg/kg to about 5 mg/kg.

As used herein, the terms “administering” and “administration” refer to any method of providing an α5β1 integrin inhibitor to a patient. Such methods are well known to those skilled in the art and include, but are not limited to, intraperitoneal injection, intravenous injection, or intraarterial injection. Other modes of administration include, for example, subcutaneous administration and intranasal administration.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein the term “hypoperfusion” means decreased blood flow through a tissue or organ. Hypoperfusion may be caused by several occurrences including a decrease in general blood pressure, damage to the vasculature of a tissue or organ system, or occlusion, in part or whole, of the vasculature in a tissue or organ. Occlusion of the vasculature can be systemic or isolated to particular veins or arteries. Furthermore, occlusion of vasculature may be partial, meaning the vasculature is only partially blocked and the rate of blood flow is reduced. Occlusion of vasculature may also be full, meaning the vasculature is fully blocked and the rate of blood flow is approximately zero, even if for a moment. Occlusion of vasculature may also be chronic or acute.

In some embodiments of the presently-disclosed subject matter, the α5β1 integrin inhibitor can be administered at a dose of about 1 mg/kg. In some embodiments, the α5β1 integrin inhibitor can be administered at a dose of about 0.1, 0.2, 0.3, 0.4, 0.5, 0.6, 0.7, 0.8, 0.9, or 1 mg/kg. In some embodiments, the α5β1 integrin inhibitor can be administered at a dose of about 1, 2, 3, 4, 5, 6, 7, 8, 9, or 10 mg/kg. In some embodiments a patient is administered one dose daily, one dose every other day, 6 doses weekly, 5 doses weekly, 4 doses weekly, 3 doses weekly, 2 doses weekly, or 1 dose weekly.

### EXAMPLES

Materials & Methods

Wildtype (WT; C57Bl/6, male, 3 months old), α5KO (male, 3 months old), and DB (Leprdb/db, male, 3 and 8 months old) mice will be used for the following studies. Groups will include: sham/BCAS surgery, vehicle/ATN-161 treatment, 3 time points, 8 mice/group, and 4 terminal outcome measures (IHC, WB, FITC-Dextran stain, Di I stain). All animal work will be conducted under the University of Kentucky IACUC approval as per an amendment to the existing 2 R01 NS065842-08 approved protocol. Using WT and α5KO mice at 3 months of age allows comparisons between the present data and those from other studies which use mice of similar age.

Mice (WT, α5KO, DB) are subjected to either sham or BCAS (0.18 mm ID coils) surgery. Euthanasia and brain tissue extraction, and processing for immunohistochemistry or western blot analysis (see below) will occur at early (14 days) or late (30 and 60 days) time points following BCAS. This will allow correlation of α5β1 integrin expression levels with the progression of brain injury over time, whether it could potentially contribute to this pathology early and/or late, and determine whether this is influenced by the diabetic phenotype prior to overt cerebrovascular pathology.

Stroke Surgery

Mice underwent transient tandem ipsilateral common carotid artery/middle cerebral artery (CCA/MCA) occlusion for 60 minutes as previously published, followed by reperfusion for 1-3 days. Briefly, a small burr hole is created directly over the M2 branch of the MCA, and a 0.0005 inch metal filament is placed under the artery on both sides of the burr hole. The CCA was then isolated and occluded using an aneurysm clip. Blood flow was determined with the Laser Doppler Perfusion Monitor (Perimed, Ardmore, Pa., USA). Animals that received less than 80% occlusion rate from baseline (i.e. pre-occlusion) were excluded from analysis. Sham animals underwent the listed procedure without vascular occlusion. All mice were allowed to recover to post-stroke day (PSD)3, during which daily body weights were measured.

Physiological Measurements

Immediately following reperfusion, heart, rate, pulse distension, and body temperature were recorded using MouseOx (Starr Life Science, Holliston, Mass., USA). After 5 minutes, sterile PBS (Vehicle) or ATN-161 was injected via IP (as described below); mice were continually observed for 15 minutes post reperfusion.

Animal Treatments

Animals received an IP injection of either Vehicle (sterile PBS) or ATN-161 (lmg/kg, Medkoo Biosciences, Morrisville, N.C., USA) immediately after reperfusion, PSD1 and on PSD2. Ischemic stroke was confirmed on 2 mm sections stained with 2,3-triphenyltetrazolium chloride (TTC; BD, Sparks, M D, USA) or Magnetic Resonance Imaging on PSD3. Accounting for edema volume, infarct size based on TTC images was analyzed with Image J (NH) software as:

$$Infarct volume = infarct \times ( \frac{Contralateral Hemisphere}{Ipsilateral Hemisphere} )$$

Animals were excluded from the study if the CCA and/or MCA was punctured during wire and clamp placement or removal.

Magnetic Resonance Imaging (MRI)

PSD3 animals were anesthetized with isoflurane and placed on a heated bed where body temperature and breathing rate were carefully monitored throughout the scan. All images were performed by a Bunker/Siesman 7Tesla MR in the University of Kentucky Magnetic Resonance Imaging and Spectroscopy Center. T1 and T2 weighted images of the brain were obtained in the transverse plane without intravenous contrast. Additionally, Apparent Diffusion Coefficient (ADC) images were produced in the transverse plane using Diffusion Tensor Imaging (DTI). All images were analyzed with ITK-SNAP (www.itksnap.org) by a blinded, independent neuroradiologist. Edema and infarct volume were determined independently by software based manual segmentation. T2 weighted images and ADC images were examined by an experienced neuroradiologist. Areas of abnormally high signal intensity on the T2 weighted images, representing edema, and areas of abnormally low signal intensity on the ADC images, representing acute infarction, were visually identified and manually outlined or segmented using computer software.

Tissue Histology and Immunohistochemistry

On PSD3, animals were systemically perfused with PBS (Phosphate Buffered Solution, 1×) for 5 minutes. Brains were removed, flash frozen in liquid nitrogen, and stored at −20 C until use. The brains were coronally sectioned by cryostat at 20 μm and mounted (four per slide, 2 mm separating each section) for staining. Negative controls (no primary antibody) were run in tandem with each target to determine thresholding values.

IgG Immunohistochemistry

Sections were fixed in 10% phosphate-buffered formalin. After washing, IgG 568 biotinylated antibody (1:500, Thermo-Fisher Scientific) in 1% bovine serum albumin was applied to the tissue for 2 hours. Slides were then cover slipped (Vector Labs) for imaging on a Nikon Eclipse Ti and software (Nikon, Melville, N.Y., USA). Image J was used to isolate and count IgG positive pixels. Three separate 20× images per core region were taken per section and averaged.

Immunohistochemistry

Sections were fixed in methanol:acetone (1:1), followed by blocking buffer (5% bovine serum albumin (Fisher Scientific) in PBS with 0.1% Triton-X). Sections were incubated in primary antibody overnight at 4 C (anti-rabbit PECAM at 1:100, Fisher Scientific; anti-rat CD49e at 1:250, BD Pharminagen; anti-rat CD45 at 1:200, ThermoFisher Scientific, anti-rabbit Claudin-5 at 1:200, Abcam, anti-rabbit MMP-9 at 1:100, Abcam, and anti-rabbit fibronectin at 1:100, Abcam). Sections were washed and placed in fluorophore-conjugated secondary antibody, followed by NucBlue cell nucleus counterstain (Fisher Scientific) at room temperature (Vector Labs, 1:250), then cover slipped (Vector Labs) and imaged on a Nikon Eclipse Ti and software (Nikon, Melville, N.Y., USA). Image J was used to quantify all Immunofluorescence positive pixels. CD45: One 4× image per all four sections were taken and averaged. Claudin-5 and CD49e: In the core, one 10× image per all four sections was taken and averaged. In the peri-infarct, two 10× images per all four sections were taken and averaged.

Behavioral Testing

Stroked mice underwent an 11-point behavioral neurological score behavioral assessment on PSD0 (Baseline prior to drug administration), PSD1, 2, and 3 to assess the following behavioral metrics: level of consciousness (LOC), gaze (G), visual field (VF), sensorimotor response (SR), grip strength and endurance/paralysis paw hang (PPH). LOC was determined as natural movement prior to any cage disturbance. A severity score of 0-2 was assigned, 0 being alert and active, 1 alert upon stimulation, and 2 for being hunched, unstimulated, and loss of grooming. Gaze was assessed by waving an object in front of each eye without disturbing the whiskers. Based on a severity score of 0-1, 0 was given if they recognized the sign by turning their head, and a 1 was given if no response was noted. VF was assessed holding the mouse vertically next to a platform by the tail (both right and left sides). A score of 0 was given if the mouse reached for the platform, and a 1 for failure to do so. SR was scored by pressing each paw in turn to elicit vocalization, paw retraction, or jumping as a reaction to pressure. A score of 0 was given for exhibiting a reaction, and a 1 for no reaction. Finally, PPH was scored by a paw hang test. Only front paws were used for gripping of a rod while being supported by a hold on the tail for 60 seconds. A score of 0 was given for completion of the full 60 seconds, a 1 for any dropping of a paw without full loss of contact, and a 2 for a fall. The total scores are tallied at the conclusion of the testing to assess overall function.

Gene Expression

On PSD3, brains were removed and sectioned at 2 mm where the ipsilateral cortical tissue and corresponding contralateral cortical tissue were isolated. All sections per animal were combined and homogenized in Trizol (Life Technologies). RNA extraction was performed according to the RNA extraction kit provided by the manufacturer (Life Technologies). RNA was converted to cDNA using a high capacity cDNA reverse transcriptase kit (Applied Bioscience, Grand Island, N.Y., USA). 18S (control; Mm03928990_g1), collagen IV (Mm00483669_m1), IL-1β (Mm00434228_m1), CXCL12 (Mm00445553_m1), and MMP9 (Mm00442991_m1) Taqman genes were analyzed using Real-time PCR (ViiA7, Life Technologies). Data was analyzed as fold change comparing vehicle and ATN-161 treated mice as a % control (sham).

Blinding and Randomization

All experiments and data analysis were conducted in a blinded and randomized fashion using an online randomization generator.

Statistical Analysis

All measureable values are presented as mean±standard deviation (SD). Sample size was determined for 80% effect by power analysis. Comparisons between Vehicle and ATN-161 treatment were performed using a Student's t-test. For multiple comparisons, a one or two-way repeated measures ANOVA was used with the posthoc Bonferroni test. Significance is determined as p*≤0.05, p**≤0.01, p***≤0.001, and p****≤0.0001. Animals were excluded based on death during surgery or animals not receiving all three injections. Outliers were determined by the interquartile range (IQR).

Results

### Example 1: ATN-161 Administration as a Treatment for Vascular Dementia

At 14 days post-stenosis increased BBB permeability (Evans blue leakage), increased glial cell inflammation, and increased cell proliferation within the striatum and cortex. Interestingly, it was also observed a significant increase in endothelial cell α5β1 integrin expression at 14 days post-stenosis (FIG. 3A-FIG. 3D).

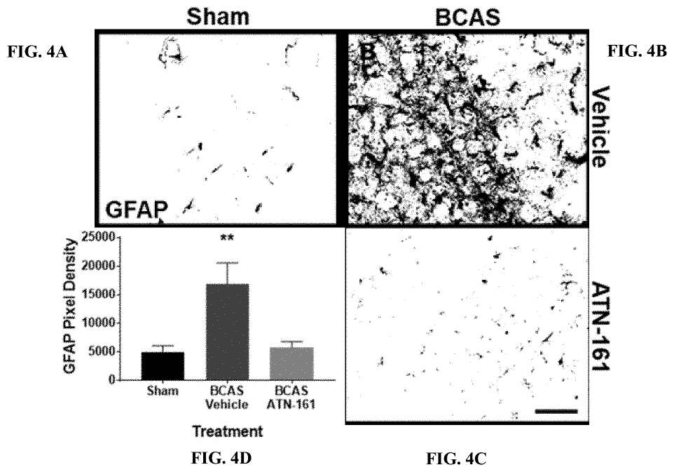

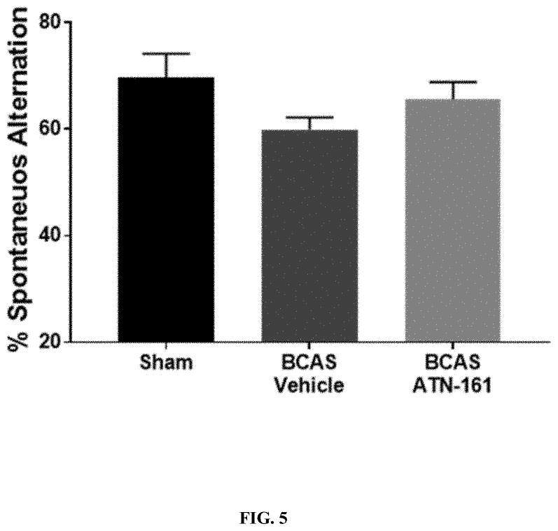

Preliminary data show that inhibition of α5β1 integrin with ATN-161 treatment in the BCAS mouse model not only decreases α5β1 integrin expression compared to controls (FIG. 3A-FIG. 3D), but also decreases inflammation (astrocyte activation) within the white matter (FIG. 4A-FIG. 4D), and improves cognitive outcome (y-maze) as compared to controls (FIG. 5).

In a previously published study there was not noted significant changes in either BBB integrity or brain α5β1 integrin prior to day 14 after BCAS. However, in initial BCAS ATN-161 study (FIGS. 3A-3D, FIGS. 4A-4D and FIG. 5), ATN-161 treatment began immediately after BCAS and then every other day until day 12 with the intent of maximizing the potential to hit a ‘BCAS therapeutic window’ for this ‘proof-of-concept’ study. However, as this acute post-hypoperfusion therapeutic approach is less clinically relevant for chronic brain hypoperfusion injury, and likely loads the brain with an α5β1 integrin inhibitor for days prior to significant α5β1 integrin expression/upregulation.

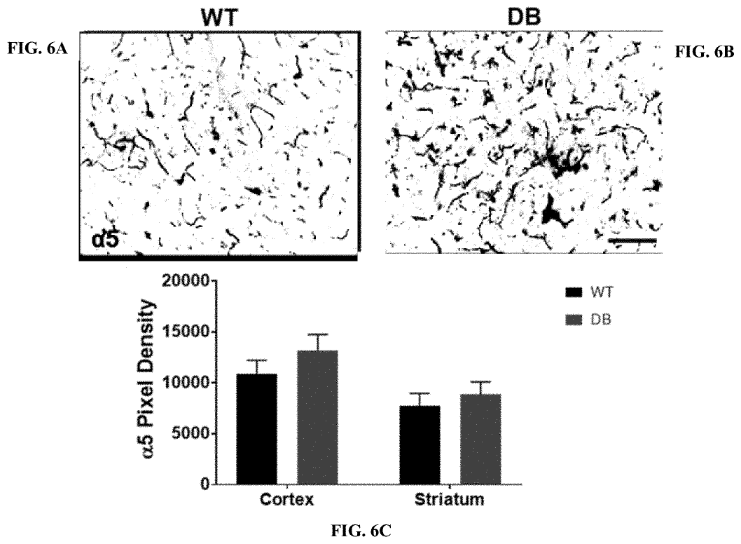

Studies further indicate that increased α5β1 integrin expression does occur in the brains of 8 month old DB mice (not subjected to BCAS) compared to controls (FIG. 6A-FIG. 6C).

### Example 2: ATN-161 Administration for the Treatment of Stroke

ATN-161 Treated Mice Show Reduced Infarct Size Following Experimental Ischemic Stroke

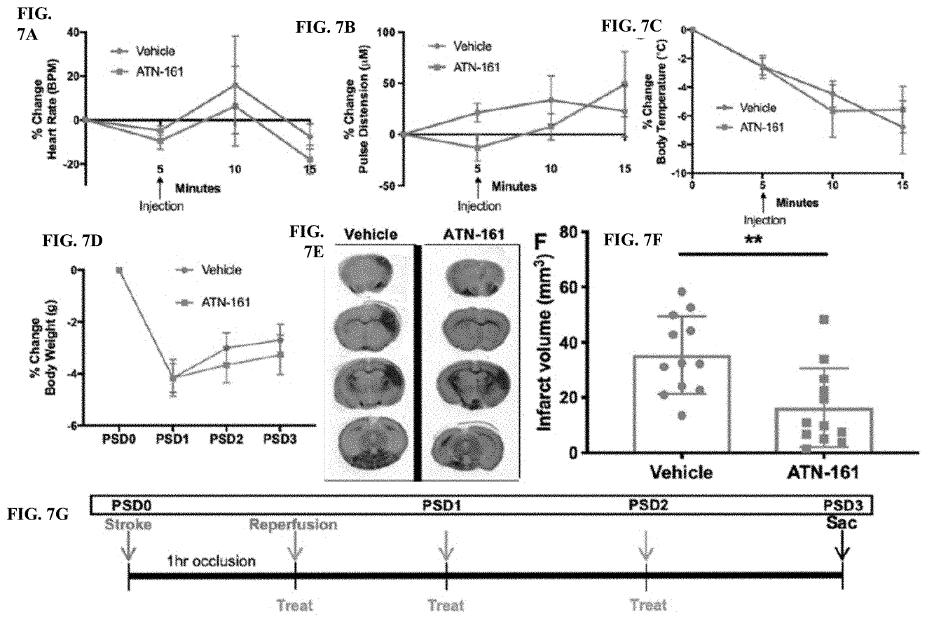

First, it was determined whether acute, post-reperfusion IP ATN-161 administration was safe immediately following experimental ischemic stroke. ATN-161 treated animals did not show any differences in heart rate (FIG. 7A), pulse distension (FIG. 7B), body temperature (FIG. 7C), and body weight (FIG. 7D) following ischemic stroke compared to Vehicle treated stroked controls.

Once acute safety was determined, stroked mice were IP administered Vehicle or ATN-161 immediately after reperfusion, on PSD1, and PSD2 (FIG. 7G). By TTC assessment, mice treated with ATN-161 had smaller infarcts when including edema, on PSD3 (FIG. 7E-F; p=0.0004; ATN-161 16.3714.25, n=12; Vehicle 35.4114.04, n=12).

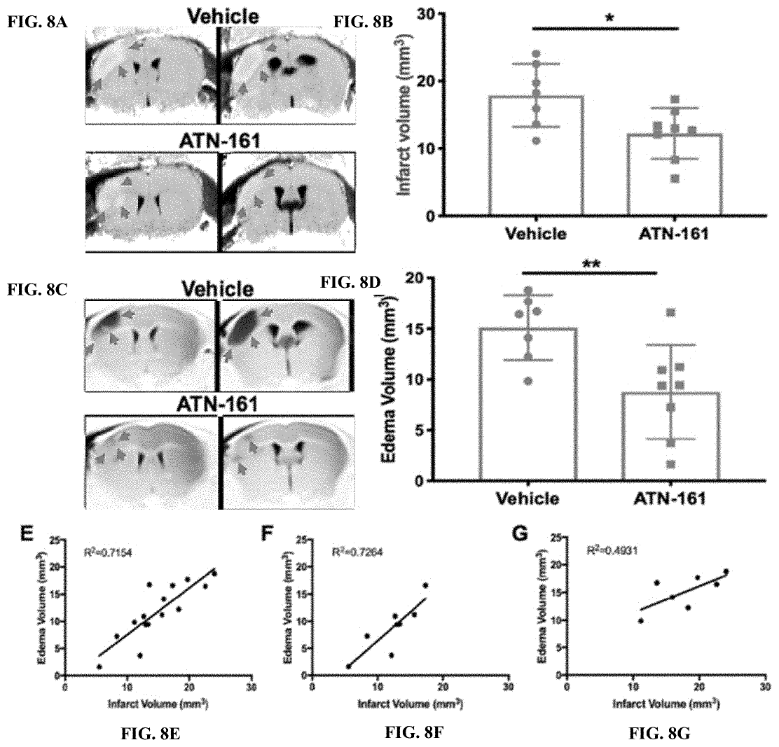

To further validate ATN-161 effects on infarct volume in a translationally relevant manner, stroked mice underwent DTI with ADC imaging on PSD3. Infarct volume, absent edema volume, was again shown to be reduced in ATN-161 treated mice (FIG. 8A-B; p=0.0220; Vehicle 17.9±4.677, n=7; ATN-161 12.24±3.758, n=8). Collectively the TTC and ADC analysis shows a decrease in infarct volume following ATN-161 treatment.

ATN-161 Administration Reduces Edema Following Experimental Ischemic Stroke

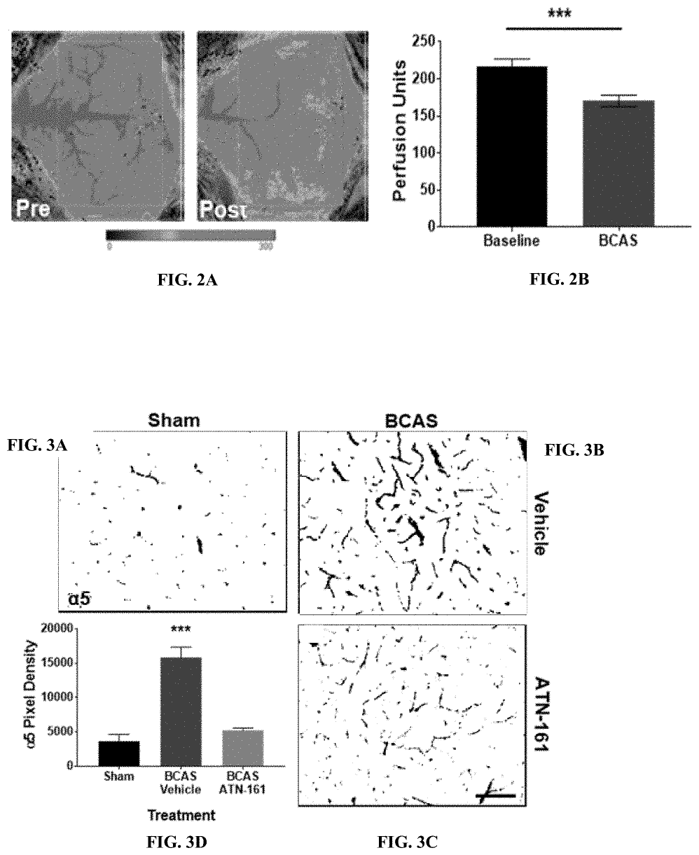

In addition to infarct volume, the extent of post-stroke edema with T2-weighted MRI separate from infarct volume determination was also determined. Imaging showed a reduction in edema in ATN-161 treated mice (FIG. 2C-D; p=0.0097; Vehicle 15.12±3.206, n=7; ATN-161 8.777±4.658, n=8). Comparison of all treatments collectively revealed a positive correlation between infarct and edema volume (All treatments p=0.0001 R²=0.7154). However, the ATN-161 treated mice (ATN-161 p=0.0072, R²=0.0072), but not the Vehicle group (Vehicle p=0.0786, R²=0.4931), showed a linear correlation relationship between infarct volume and edema volume (FIG. 8E-G, respectively). This suggests that upon the inhibition of integrin α5β1, edema occurrence is likely a direct consequence of infarct size.

ATN-161 Treated Mice have Better Functional Outcomes

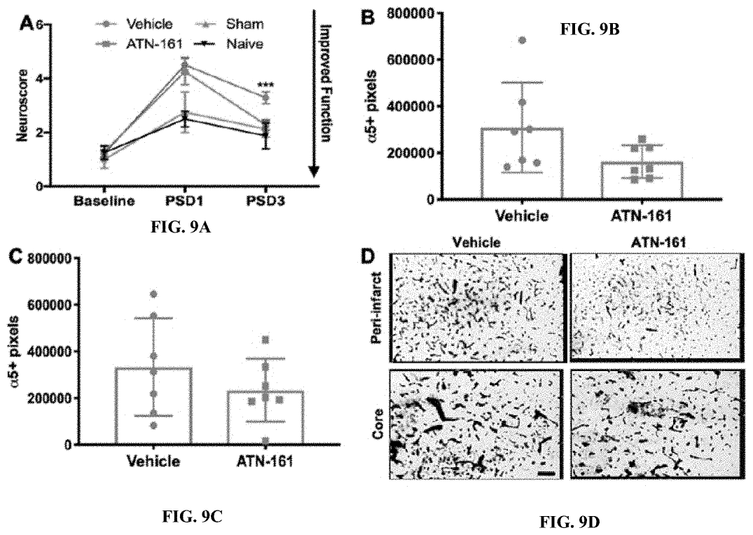

ATN-161 treated mice showed less functional deficit on PSD3 by Neuroscore (FIG. 9A; p=0.0002; Vehicle 2.286±0.8284, n=14; ATN-161 3.286±0.7263, n=14; 95% Cl −0.07218 to 0.4636), while there is no difference on PSD1.

ATN-161 Reduces α5β1 Expression Following Ischemic Stroke

α5β1 integrin expression decreased at PSD3 with ATN-161 administration in both the peri-infarct (FIG. 9B,D; p=0.0835, Vehicle 308484±53485, n=7; ATN-161 162166±23348, n=7) and core (FIG. 9C,D; p=0.3138, Vehicle 332114±209410, n=7; ATN-161 233111±134950, n=7) regions of the stroke, although these differences were not significant.

ATN-161 Reduces BBB Permeability Following Experimental Ischemic Stroke

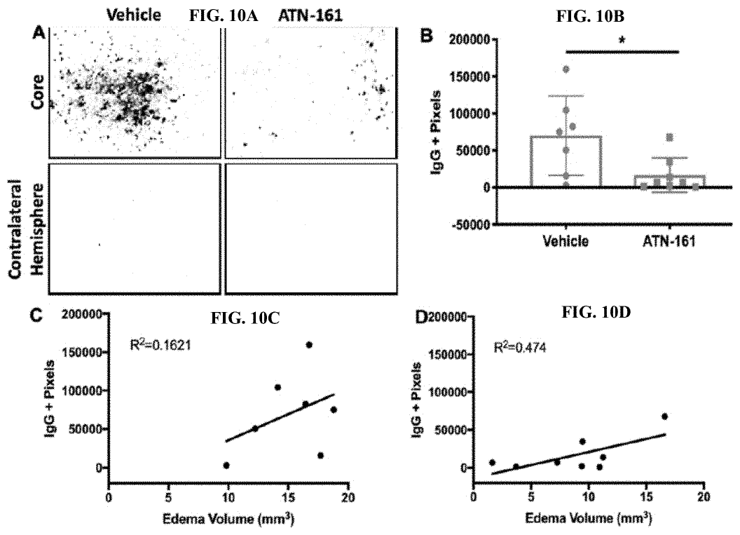

Since α5KO mice showed a stabilized BBB after experimental stroke¹⁴, BBB stability via brain IgG extravasation following ATN-161 administration was next determined. ATN-161-treated animals had significantly less parenchymal IgG extravasation in the ischemic core (FIG. 10A-B; p=0.0234; Vehicle 70125±53485, n=7; ATN-161 16753±23348, n=8). Interestingly, ATN-161 treated mice did experience a correlation between IgG extravasation (i.e. degree of BBB opening) and edema (FIG. 10D; p=0.0590, R²=0.474). This correlation disappeared in Vehicle treated mice (FIG. 10C; p=0.3705, R²=0.1621). However, IgG extravasation and infarct volume did not correlate in either Vehicle (p=0.9732, R²=0.0002498) or ATN-161 (p=0.1309, R²=0.3377) treated mice. This suggests a direct role for integrin α5β1, and its blockade, in determining the amount of edema following ischemic stroke due to regulating dysfunction of the BBB. Collectively, reduced infarct volume may be a consequence of inhibition of integrin α5β1 through a less permeable BBB, and thus reduced edema.

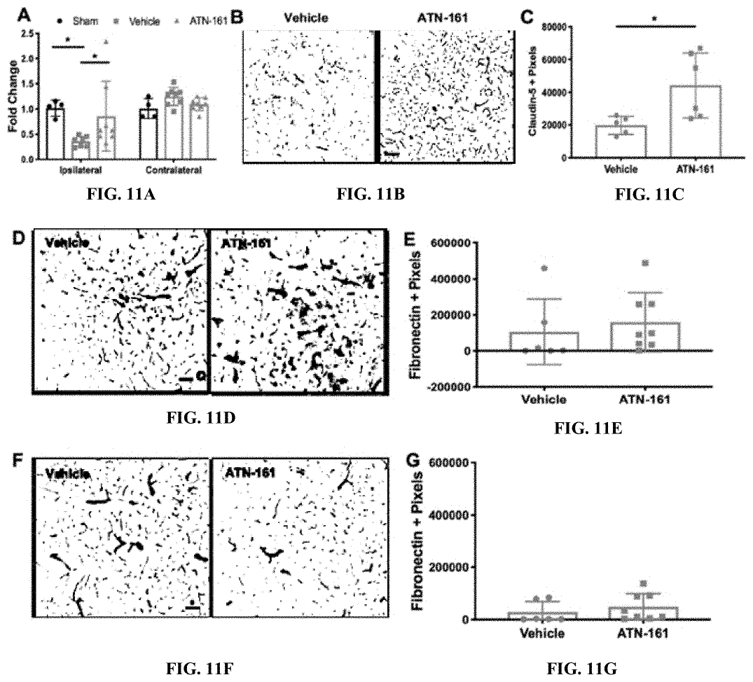

Next, to determine integrin α5β1's role in cerebrovascular pathology after stroke, changes in vascular ECM proteins, proteinases that degrade the ECM, and TJs were analyzed. On PSD3, Vehicle treated mice exhibit decreased collagen IV (an abundant ECM protein that is essential to BBB health) transcription compared to sham and ATN-161 treated animals (FIG. 11A; p=0.0115; Sham 1.018±0.1573, n=4; Vehicle 0.3543±0.1013, n=7; ATN-161 0.8563±0.6898, n=8; 95% Cl −0.6093 to −0.1379), while ATN-161 treated animals were recovered to sham levels (p=0.7272). ATN-161 treatment resulted in an increase of claudin-5 expression (immunofluorescence) in the core (FIG. 11B-C; p=0.0270; Vehicle 19830±5538, n=5; 44220±19878, n=6). However, there was no change in the ECM protein fibronectin in either the core (FIG. 11D-E; p=0.5819; Vehicle 106153±183136, n=6; ATN-161 158938164906, n=8), or peri-infarct (FIG. 11F-G; p=0.4665; Vehicle 28782±40979, n=6; ATN-161 4802816730, n=8) region between Vehicle and ATN-161 treated animals.

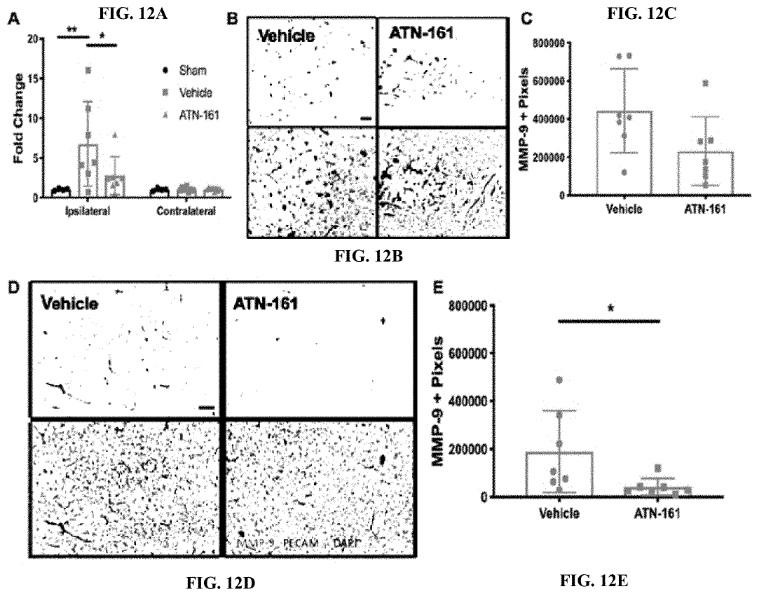

Finally, it was determined that the transcription of MMP-9 (an ECM proteinase that typically increases following stroke) is increased in Vehicle treated animals compared to Sham and ATN-161 treated mice (FIG. 12A; p=0.0028; Sham 1.018±0.1759, n=4; Vehicle 6.761±5.321, n=7; ATN-161 2.757±2.401, n=7; 95% Cl 0.7299 to 4.232), though no significance difference was noted between Vehicle and ATN-161 treated mice (p=0.5238). The protein expression of MMP-9 through immunohistochemical staining was next determined. While the decrease of MMP-9 in ATN-161 treated mice remained consistent with qPCR results in the peri-infarct (FIG. 12D-E; p=0.0450; Vehicle 189516±170441, n=7; ATN-161 42227±180171, n=7) with a significant decrease in MMP-9 in ATN-161 treated mice when compared with vehicle treated mice, no significant difference was found between Vehicle and ATN-161 treated mice in the core of the infarct (FIG. 12B-C p=0.0734; Vehicle 443691±220951, n=6; ATN-161 232262±68098, n=7). Collectively, inhibition of α5β1l by ATN-161 stabilizes the vasculature at the site of injury (increased collagen IV, decreased MMP-9, and increased claudin-5 expression) and results in a more intact BBB (less IgG extravasation) following ischemic stroke.

ATN-161 Reduces the Inflammatory Response in the Infarcted Region

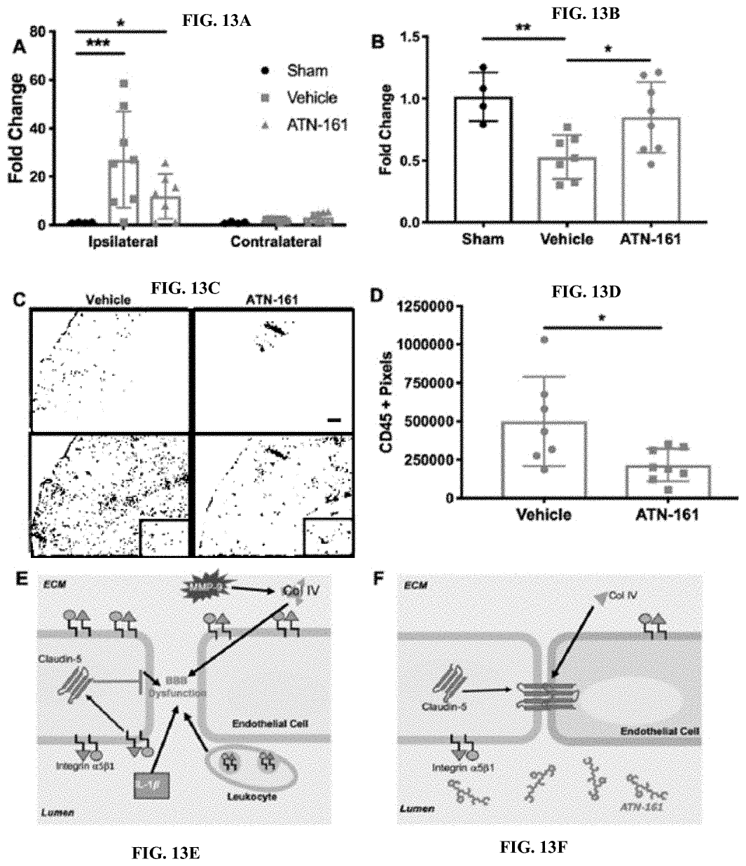

Inflammatory cascades occur acutely and last for up to a week after stroke. This involves an increase in inflammatory cytokines and neutrophil infiltration, all localized to the core site of injury. Furthermore, integrins have been implicated in many inflammatory processes. To determine ATN-161's potential effects on post-stroke inflammation, ATN-161's effects on the expression of the master inflammatory cytokine, IL-1β, which is well known to be upregulated after experimental ischemic stroke were determined. Indeed, IL-1β transcription is increased in the Vehicle treated mice compared to sham (FIG. 13A; p=0.0006; Vehicle 27.04±19.96, n=8; Sham 1.023±0.1666, n=4; 95% Cl 4.209 to 18.30). ATN-161 administration, significantly reduced (p=0.0192; ATN-161 11.89±9.235, n=7) IL-1β expression. Investigating the chemokine ligand 12 (CXCL12), previously shown to be protective following ischemic stroke²⁰, Vehicle treated mice had reduced transcription of CXCL12 compared to sham and ATN-161 (FIG. 13B; p=0.0022; Vehicle 0.5271±0.1779, n-7; Sham 1.015±0.1964, n=4; ATN-161 0.8488±0.2846, n=8), while ATN-161 treated mice recovered CXCL12 to sham levels.

Additionally, integrins have not only been implicated in cytokine and chemokine expression, but also in migration of inflammatory neutrophils to the site of injury, a mechanism highly implicated in edema and expansion of the core. Even though neutrophils have the highest significance of all leukocytes trafficking to the site of injury, all categories of leukocytes have a role. Because of this, the effect of ATN-161 on CD45, a pan leukocyte marker, at PSD3 via immunofluorescence was determined. Again, fewer infiltrating cells were found in the ipsilateral stroked brain parenchyma with ATN-161 treatment (FIG. 13C-D; p=0.0225; Vehicle 499674±290485, n=7; ATN-161 215451±106062, n=8). Taken together, inhibition of integrin α5β1 with ATN-161 reduces the inflammatory load following ischemic stroke.

ATN-161 has been previously described as an anti-angiogenic therapeutic, where the safety and efficacy of ATN-161 have been described in cancer pre-clinical research and clinical trials. ATN-161 treatment led to decreases in infarct volume, edema, and functional deficit through inhibition of the integrin α5β1 following ischemic stroke. Post-stroke ATN-161 administration stabilizes the BBB as evidenced by decreased IgG extravasation, stabilization of collagen IV and claudin-5 expression, as well as significantly fewer infiltrated leukocytes, and decreases in both MMP-9, CXCL12, and IL-1β mRNA expression through inhibition of integrin α5β1. The effects of integrin α5β1 on cerebrovasculature following ischemic stroke, are summarized in FIGS. 13E and 13F. The present invention is the first to demonstrate the therapeutic efficacy of inhibiting integrin α5β1 in ischemic stroke.

Importantly, ATN-161-mediated effects on function only occurred after all three injections were administered. This suggests a need for continuous acute and subacute inhibition of integrin α5β1 following ischemic stroke to inhibit any intermittent vascular remodeling that may occur, a major role of integrin α5β1. acute and subacute post-stroke doses of ATN-161 both inactivates and reduces the expression of integrin α5β1 in the context of this potential positive feedback loop.

“Flooding the system” with ATN-161 is clinically possible as previous clinical trials (Phase I and II for tumorigenesis) showed limited side effects (dry mouth, cellulitis, and paresthesia at 1 mg/kg 10-minute infusions for one out of four patients). The three-dose administration strategy was based on the acute upregulation of integrin α5β1. In addition, the dose administration strategy was based on previous research by Donate et al. which showed that ATN-161 at doses equivalent to 1-5 mg/kg produced the most significant reduction in activity (angiogenesis and tumor growth).

There was reduced edema volume and less IgG extravasation upon ATN-161 administration, in ATN-161 that was corroborated with strong correlations on ATN-161 treated mice that was lost with Vehicle treated mice.

Inhibition of integrin α5β1 via ATN-161 also resulted in reduced IL-1β expression and fewer infiltrating CD45+ leukocytes. Leukocytes (neutrophils, monocytes, macrophages, etc.) are highly implicated in increasing edema through adhesion of 32 integrin. Decreased leukocytic MMP-9s are associated with reduced BBB leakage, reduced collagen IV proteolysis, decreased neutrophil infiltration, and a relative absence of hemorrhagic transformation. Additionally, conservation of CXCL12 transcription post-stroke with ATN-161 administration was observed. The chemokine, CXCL12, has been shown to be protective in hypoxia pre-conditioning by anchoring astrocytic endfeet to BBB proteins, thus reducing leukocytic infiltration and increased BBB permeability. The results suggest that integrin α5β1 may play a significant role in regulating claudin-5 expression in endothelial cells, while also regulating neutrophilic infiltration and the associated mechanisms of BBB breakdown.

As integrin α5β1 is pro-angiogenic, long-term inhibition may prove to be deleterious as previous studies have shown that patients with increased vascular density tend to have better stroke outcomes.

Intriguingly, the present results are seemingly in direct contrast to a recent study demonstrating that administration of a single IP dose of the small peptide α5β1 integrin agonist. PHSRN, one amino acid different than ATN-161, PHSCN, 24 hours after rat transient MCAO was both neuroprotective, proangiogenic and increased neurogenesis (Wu et al.).

### REFERENCES

Each of the following references is herein incorporated by reference in their entirety.
1. Lee B, Clarke D, Al Ahmad A, Kahle M, Parham C, Auckland L, et al. Perlecan domain V is neuroprotective and proangiogenic following ischemic stroke in rodents. The Journal of clinical investigation. 2011; 121(8):3005-23.
2. Clarke D N, Al Ahmad A, Lee B, Parham C, Auckland L, Fertala A, et al. Perlecan Domain V induces VEGf secretion in brain endothelial cells through integrin alpha5beta1 and ERKdependent signaling pathways. PloS one. 2012; 7(9):e45257.
3. Li L, Welser-Alves J, van der Flier A, Boroujerdi A, Hynes R O, Milner R. An angiogenic role for the alpha5beta1 integrin in promoting endothelial cell proliferation during cerebral hypoxia. Experimental neurology. 2012; 237(1):46-54.
4. van der Flier A, Badu-Nkansah K, Whittaker C A, Crowley D, Bronson R T, Lacy-Hulbert A, et al. Endothelial alpha5 and alphav integrins cooperate in remodeling of the vasculature during development. Development. 2010; 137(14):2439-49.
5. Roberts J, de Hoog L, Bix G J. Mice deficient in endothelial alpha5 integrin are profoundly resistant to experimental ischemic stroke. J Cereb Blood Flow Metab. 2017; 37(1):85-96.
6. Sandoval K E, Witt K A. Blood-brain barrier tight junction permeability and ischemic stroke. Neurobiol Dis. 2008; 32(2):200-19.
7. Prakash R, Carmichael S T. Blood-brain barrier breakdown and neovascularization processes after stroke and traumatic brain injury. Curr Opin Neurol. 2015; 28(6):556-64.
8. Levine D A, Langa K M. Vascular cognitive impairment: disease mechanisms and therapeutic implications. Neurotherapeutics. 2011; 8(3):361-73.
9. Shibata M, Ohtani R, Ihara M, Tomimoto H. White matter lesions and glial activation in a novel mouse model of chronic cerebral hypoperfusion. Stroke. 2004; 35(11):2598-603.
10. Toyama K, Koibuchi N, Uekawa K, Hasegawa Y, Kataoka K, Katayama T, et al. Apoptosis signal-regulating kinase 1 is a novel target molecule for cognitive impairment induced by chronic cerebral hypoperfusion. Arteriosclerosis, thrombosis, and vascular biology. 2014; 34(3):616-25.
11. Miyamoto N, Pham L D, Maki T, Liang A C, Arai K. A radical scavenger edaravone inhibits matrix metalloproteinase-9 upregulation and blood-brain barrier breakdown in a mouse model of prolonged cerebral hypoperfusion. Neuroscience letters. 2014; 573:40-5.
12. Hattori Y, Enmi J, Iguchi S, Saito S, Yamamoto Y, Nagatsuka K, et al. Substantial Reduction of Parenchymal Cerebral Blood Flow in Mice with Bilateral Common Carotid Artery Stenosis. Scientific reports. 2016; 6:32179.
13. Holland P R, Searcy J L, Salvadores N, Scullion G, Chen G, Lawson G, et al. Gliovascular disruption and cognitive deficits in a mouse model with features of small vessel disease. Journal of cerebral blood flow and metabolism: official journal of the International Society of Cerebral Blood Flow and Metabolism. 2015; 35(6):1005-14.
14. Roberts J M, Maniskas M E, Bix G J. Bilateral carotid artery stenosis causes unexpected early changes in brain extracellular matrix and blood-brain barrier integrity in mice. PLoS One. 2018; 13(4):e0195765.
15. Colinas O, Moreno-Dominguez A, Zhu H L, Walsh E J, Perez-Garcia M T, Walsh M P, et al. alpha5-Integrin-mediated cellular signaling contributes to the myogenic response of cerebral resistance arteries. Biochem Pharmacol. 2015; 97(3):281-91.
16. Akinyemi R O, Mukaetova-Ladinska E B, Attems J, Ihara M, Kalaria R N. Vascular risk factors and neurodegeneration in ageing related dementias: Alzheimer's disease and vascular dementia. Curr Alzheimer Res. 2013; 10(6):642-53.
17. Ott A, Stolk R P, van Harskamp F, Pols H A, Hofman A, Breteler M M. Diabetes mellitus and the risk of dementia: The Rotterdam Study. Neurology. 1999; 53(9):1937-42.
18. Nishijima Y, Akamatsu Y, Yang S Y, Lee C C, Baran U, Song S, et al. Impaired Collateral Flow Compensation During Chronic Cerebral Hypoperfusion in the Type 2 Diabetic Mice. Stroke. 2016; 47(12):3014-21.
19. Kwon K J, Lee E J, Kim M K, Kim S Y, Kim J N, Kim J O, et al. Diabetes augments cognitive dysfunction in chronic cerebral hypoperfusion by increasing neuronal cell death: implication of cilostazol for diabetes mellitus-induced dementia. Neurobiol Dis. 2015; 73:12-23.
20. Santiago E, Delevatti R S, Bracht C G, Netto N, Lisboa S C, Vieira A F, et al. Acute glycemic and pressure responses of continuous and interval aerobic exercise in patients with type 2 diabetes. Clin Exp Hypertens. 2018; 40(2):179-85.
21. Zuloaga K L, Johnson L A, Roese N E, Marzulla T, Zhang W, Nie X, et al. High fat diet-induced diabetes in mice exacerbates cognitive deficit due to chronic hypoperfusion. J Cereb Blood Flow Metab. 2016; 36(7): 1257-70.
22. Stranahan A M, Hao S, Dey A, Yu X, Baban B. Blood-brain barrier breakdown promotes macrophage infiltration and cognitive impairment in leptin receptor-deficient mice. J Cereb Blood Flow Metab. 2016; 36(12):2108-21.
23. Hummel K P, Dickie M M, Coleman D L. Diabetes, a new mutation in the mouse. Science. 1966; 153(3740): 1127-8.
24. Niedowicz D M, Reeves V L, Platt T L, Kohler K, Beckett T L, Powell D K, et al. Obesity and diabetes cause cognitive dysfunction in the absence of accelerated beta-amyloid deposition in a novel murine model of mixed or vascular dementia. Acta Neuropathol Commun. 2014; 2:64.
25. Liao Y J, Ueno M, Nakagawa T, Huang C, Kanenishi K, Onodera M, et al. Oxidative damage in cerebral vessels of diabetic db/db mice. Diabetes Metab Res Rev. 2005; 21(6):554-9.
26. Khalili P, Arakelian A, Chen G, Plunkett M L, Beck I, Parry G C, et al. A non-RGD-based integrin binding peptide (ATN-161) blocks breast cancer growth and metastasis in vivo. Mol Cancer Ther. 2006; 5(9):2271-80.
27. Chidlow J H, Jr., Langston W, Greer J J, Ostanin D, Abdelbaqi M, Houghton J, et al. Differential angiogenic regulation of experimental colitis. Am J Pathol. 2006; 169(6):2014-30.
28. Beckmann N, Stirnimann R, Bochelen D. High-resolution magnetic resonance angiography of the mouse brain: application to murine focal cerebral ischemia models. J Magn Reson. 1999; 140(2):442-50.
29. Krucker T, Schuler A, Meyer E P, Staufenbiel M, Beckmann N. Magnetic resonance angiography and vascular corrosion casting as tools in biomedical research: application to transgenic mice modeling Alzheimer's disease. Neurol Res. 2004; 26(5):507-16.
30. Li H Y, Wang X C, Xu Y M, Luo N C, Luo S, Hao X Y, et al. Berberine Improves Diabetic Encephalopathy Through the SIRT1/ER Stress Pathway in db/db Mice. Rejuvenation Res. 2017.
31. Silvers J M, Harrod S B, Mactutus C F, Booze R M. Automation of the novel object recognition task for use in adolescent rats. J Neurosci Methods. 2007; 166(1):99-103.
32. Marques F, Sousa J C, Sousa N, Palha J A. Blood-brain-barriers in aging and in Alzheimer's disease. Mol Neurodegener. 2013; 8:38.
33. Liu P, Li Y, Pinho M, Park D C, Welch B G, Lu H. Cerebrovascular reactivity mapping without gas challenges. Neuroimage. 2017; 146:320-6.
34. Benjamin E J, Virani S S, Callaway C W, et al. Heart Disease and Stroke Statistics-2018 Update: A Report From the American Heart Association. Circulation. 2018; 137(12):e67-e492.
35. Albers G W, Goldstein L B, Hess D C, et al. Stroke Treatment Academic Industry Roundtable (STAIR) recommendations for maximizing the use of intravenous thrombolytics and expanding treatment options with intra-arterial and neuroprotective therapies. Stroke. 2011; 42(9):2645-50.
36. Albers G W, Marks M P, Kemp S, et al. Thrombectomy for Stroke at 6 to 16 Hours with Selection by Perfusion Imaging. N Engl J Med. 2018; 378(8):708-18.
37. Mendez A A, Samaniego E A, Sheth S A, et al. Update in the Early Management and Reperfusion Strategies of Patients with Acute Ischemic Stroke. Crit Care Res Pract. 2018; 2018:9168731.
38. Wang Q, Tang X N, Yenari M A. The inflammatory response in stroke. Journal of Neuroimmunology. 2007; 184(1-2):53-68.
39. Baeten K M, Akassoglou K. Extracellular Matrix and Matrix Receptors in Blood-Brain Barrier Formation and Stroke. Dev Neurobiol. 2011; 71(11): 1018-39.
40. Huveneers S, Truong H, Danen H J. Integrins: signaling, disease, and therapy. Int J Radiat Biol. 2007; 83(11-12):743-51.
41. Park E J, Yuki Y, Kiyono H, et al. Structural basis of blocking integrin activation and deactivation for anti-inflammation. J Biomed Sci. 2015; 22:51.
42. Huang Q, Chen B, Wang F, Huang H, et al. The temporal expression patterns of fibronectin and its receptors-alpha5beta1 and alphavbeta3 integrins on blood vessels after cerebral ischemia. Restor Neurol Neurosci. 2015; 33(4):493-507.
43. Tani N, Higashiyama S, Kawaguchi N, et al. Expression level of integrin alpha 5 on tumour cells affects the rate of metastasis to the kidney. Br J Cancer. 2003; 88(2):327-33.
44. Yurdagul A, Jr., Green J, Albert P, et al. alpha5beta1 integrin signaling mediates oxidized low-density lipoprotein-induced inflammation and early atherosclerosis. Arterioscler Thromb Vasc Biol. 2014; 34(7):1362-73.
45. Van Bergen T, Zahn G, Caldirola P, et al. Integrin alpha5beta1 Inhibition by CLT-28643 Reduces Postoperative Wound Healing in a Mouse Model of Glaucoma Filtration Surgery. Invest Ophthalmol Vis Sci. 2016; 57(14):6428-39.
46. Grupke S, Hall J, Dobbs M, et al. Understanding history, and not repeating it. Neuroprotection for acute ischemic stroke: from review to preview. Clin Neurol Neurosurg. 2015; 129:1-9.
47. Cianfrocca M E, Kimmel K A, Gallo J, et al. Phase 1 trial of the antiangiogenic peptide ATN-161 (Ac-PHSCN-NH(2)), a beta integrin antagonist, in patients with solid tumours. Br J Cancer. 2006; 94(11): 1621-6.
48. Yushkevich P A, Piven J, Hazlett H C, et al. User-guided 3D active contour segmentation of anatomical structures: significantly improved efficiency and reliability. Neuroimage. 2006; 31(3):1116-28.
49. Luheshi N M, Kovacs K J, Lopez-Castejon G, et al A. Interleukin-lalpha expression precedes IL-1beta after ischemic brain injury and is localized to areas of focal neuronal loss and penumbral tissues. J Neuroinflammation. 2011; 8:186.
50. Selvaraj U M, Ortega S B, Hu R, et al. Preconditioning-induced CXCL12 upregulation minimizes leukocyte infiltration after stroke in ischemia-tolerant mice. J Cereb Blood Flow Metab. 2017; 37(3):801-13.
51. Lorenz H M, Lagoo A S, Hardy K J. The Cell and Molecular Basis of Leukocyte Common Antigen (CD45)-Triggered, Lymphocyte Function-Associated Antigen-1-/Intercellular Adhesion Molecule-1-Dependent, Leukocyte Adhesion. Blood. 1994; 83(7): 1862-70.
52. Sun J, Yu L, Huang S, et al. Vascular expression of angiopoietin-1 α5β1 integrin and tight junction proteins is tightly regulated during vascular remodeling in the post-ischemic brain. Neurosci. 2017; 362: 248-256.
53. Donate F, Parry G C, Shaked Y, et al. Pharmacology of the Novel Antiangiogenic Peptide A TN-161 (Ac-PHSCN-NH2): Observation of a U-Shaped Dose-Response Curve in Several Preclinical Models of Angiogenesis and Tumor Growth. Clin Cancer Res. 2008; 14(7):2137-44.
54. Guell K, Bix G J. Brain endothelial cell specific integrins and ischemic stroke. Expert Rev Neurothera. 2014; 14(11): 1287-92.
55. Yu S W, Friedman B, Cheng Q, et al. Stroke-evoked angiogenesis results in a transient population of microvessels. J Cereb Blood Flow Metab. 2007; 27(4):755-63.
56. Nitta T, Hata M, Gotoh S, et al. Size-selective loosening of the blood-brain barrier in claudin-5-deficient mice. J Cell Biol. 2003; 161(3):653-60.
57. Osada T, Gu Y H, Kanazawa M, et al. Interendothelial claudin-5 expression depends on cerebral endothelial cell-matrix adhesion by beta(1)-integrins. J Cereb Blood Flow Metab. 2011; 31(10):1972-85.
58. Luscinaskas F W, Kansas G S, Ding H, et al. Monocyte Rolling, Arrest and Spreading on IL-4-activated Vascular Endothelium under Flow Is Mediated via Sequential Action of L-Selectin, b1-Integrins, and b2-Integrins. J Cell Biol. 1994; 125(6):1417-27.
59. Laukaitis C M, Webb D J, Donais K, et al. Differential Dynamics of α5 Integrin, Paxillin, and a-Actinin during Formation and Disassembly of Adhesions in Migrating Cells. J Cell Biol. 2001; 153(7): 1427-40.
60. Stanley P, Tooze S, Hogg N. A role for Rap2 in recycling the extended conformation of LFA-1 during T cell migration. Biol Open. 2012; 1(11):1161-8.
61. Gronholm M, Jahan F, Bryushkova E A, et al. LFA-1 integrin antibodies inhibit leukocyte alpha4beta1-mediated adhesion by intracellular signaling. Blood. 2016; 128(9):1270-81.
62. Pierini L M, Lawson M A, Eddy R J, et al. Oriented endocytic recycling of α5b1 in motile neutrophils. Blood. 2000; 95(8):2471-80.
63. Ohno T, Yamamoto G, Hayashi J I, et al. Angiopoietin-like protein 2 regulates Porphyromonas gingivalis lipopolysaccharide-induced inflammatory response in human gingival epithelial cells. PLoS One. 2017; 12(9):e0184825.
64. Rosell A, Lo E H. Multiphasic roles for matrix metalloproteinases after stroke. Curr Opin Pharmacol. 2008; 8(1):82-9.
65. Liu J, Wang Y, Akamatsu Y, et al. Vascular remodeling after ischemic stroke: mechanisms and therapeutic potentials. Prog Neurobiol. 2014; 115:138-56.
66. Navaratna D, Guo S, Arai K, et al. Mechanisms and targets for angiogenic therapy after stroke. Cell Adh Migr. 2009; 3(2):216-23.
67. Wu C C, Wang L C, Su Y T, et al. Synthetic alpha5beta1 integrin ligand PHSRN is proangiogenic and neuroprotective in cerebral ischemic stroke. Biomaterials. 2018; 185:142-54.
68. Feng Y, Mrksich M. The Synergy Peptide PHSRN and the Adhesion Peptide RGD Mediate Cell Adhesion through a Common Mechanism. Biochemistry. 2004; 43:15811-21.
69. Jiang M, Sun L, Feng D X, et al. Neuroprotection provided by isoflurane pre-conditioning and post-conditioning. Med Gas Res. 2017; 7(1):48-55.
70. Stoeltzing O, Liu W, Reinmuth N, et al. Inhibition of integrin alpha5beta1 function with a small peptide (ATN-161) plus continuous 5-FU infusion reduces colorectal liver metastases and improves survival in mice. Int J Cancer. 2003; 104(4):496-503.
71. Sui A, Zhong Y, Demetriades A M, et al. ATN-161 as an Integrin alpha5beta1 Antagonist Depresses Ocular Neovascularization by Promoting New Vascular Endothelial Cell Apoptosis. Med Sci Monit. 2018; 24:5860-73.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of treating or preventing a hypoperfusion related disease in a subject in need thereof, comprising: identifying a subject with cerebral hypoperfusion and administering an effective amount of an α5β1 integrin inhibitor to the subject.

2. The method of claim 1, wherein the hypoperfusion related disease is neuro inflammation, dementia, or cognitive impairment.

3. The method of claim 1, wherein the α5β1 integrin inhibitor is selected from: AST-161, Ac-PhScN-NH2, CRRETAWAC, or combinations thereof.

4. The method of claim 1, wherein the cerebral hypoperfusion is chronic or acute.

5. The method of claim 1 wherein the cerebral hypoperfusion fully or partially occludes vasculature.

6. The method of claim 1, wherein the cerebral hypoperfusion is caused by a stroke.

7. The method of claim 6 wherein the administration of the α5β1 integrin inhibitor is acute and/or subacute.

8. The method of claim 3 wherein the α5β1 integrin inhibitor is AST-161.

9. The method of claim 8 wherein the amount of AST-161 administered is about 1 mg/kg to about 5 mg/kg.

10. A method of decreasing α5β1 expression in the cerebrovasculature, the method comprising: administering an effective amount of an α5β1 integrin inhibitor to a subject in need thereof.

11. The method of claim 10, wherein the α5β1 integrin inhibitor is selected from: AST-161, Ac-PhScN-NH2, CRRETAWAC, or combinations thereof.

12. The method of claim 11 wherein the α5β1 integrin inhibitor is AST-161.

13. The method of claim 12 wherein the amount of AST-161 is about 1 mg/kg to about 5 mg/kg.

14. A method of preventing or decreasing the risk of developing diabetes related dementia, comprising: identifying a subject with diabetes and administering an effective amount of an α5β1 integrin inhibitor to the subject.

15. The method of claim 14, wherein the α5β1 integrin inhibitor is selected from: AST-161, Ac-PhScN-NH2, CRRETAWAC, or combinations thereof.

16. The method of claim 15 wherein the α5β1 integrin inhibitor is AST-161.

17. The method of claim 16 wherein the amount of AST-161 is about 1 mg/kg to about 5 mg/kg.
